(19) United States
(12) Patent Application Publication
(10) Pub. No.: US 2003/0125696 A1
(43) Pub. Date: Jul. 3, 2003

(54) All direction stretchable multilayer diaper

(75) Inventors: Michael T. Morman, Alpharetta, GA (US); Thomas H. Roessler, Menasha, WI (US)

Correspondence Address: Senniger, Powers, Leavitt & Roedel, One Metropolitan Square, 16th Floor, St. Louis, MO, 63102, US

(73) Assignee: Kimberly-Clark Worldwide, Inc.

(21) Appl. No.: 10/037,457

(22) Filed: Dec. 31, 2001

(51) Int. Cl. A61F 13/15

(52) U.S. Cl. 604/385.22; 604/386

(57) ABSTRACT

The present invention provides a disposable absorbent article comprising a stretchable multilayer chassis that defines an inner surface and an outer surface, an absorbent body having an inner surface and an outer surface wherein the stretchable multilayer chassis is stretchable in at least the cross-machine direction, and the absorbent body is affixed to the inner surface of the multilayer stretchable chassis such that in use, the inner surface of the absorbent body lies against the wearer and the stretchable multilayer chassis stretches about the wearer independently in at least the cross-machine direction.

### BACKGROUND OF THE INVENTION

Various techniques and materials have been employed in the construction of disposable absorbent articles such as diapers, in order to provide the user with desired levels of appearance, fit, comfort and leakage containment.

In particular, conventional diapers employ various constructions in order to increase the stretchability of the diaper to better fit the wearer and to be resilient to the different stresses imposed by the size and movements of the wearer without compromising the comfort and fit of the article on the wearer.

For example, many conventional diapers do not possess the ability to properly stretch to comfortably fit the buttocks, waist and crotch of the wearer. Some articles have typically employed stretchable components such as waist and leg elastics, elasticized panels and the like. Further, diaper designs having stretchable components may employ folded pleats in the absorbent and in the tissue wrapsheet to provide improved fit and containment.

However, diapers that utilize pleats require precise folding equipment adding to the complexity and cost of the diaper construction. In addition, the comfort of the wearer may be compromised due to the extension of the article facings which may be inhibited by the friction of the absorbent body and the outer cover sliding over each other. Further, the friction of the absorbent body and the outer cover may be exaggerated by the weight of the wearer during diaper application.

Absorbent articles may otherwise be elasticized in order to provide enhanced stretchability of the article. These absorbent articles may incorporate elastomerically stretchable outer covers, bodyside liners, absorbent bodies and the like to stretch around the user for improved appearance, fit and leakage containment. However, absorbent articles that utilize a multitude of stretchable elastomeric components in its construction may be more costly and complex as stretchable materials can be more difficult to process during manufacturing.

Stretchable diapers, whether elastic, extensible, or both, are generally a tradeoff where greater comfort and fit is achieved by the use of more costly materials that are more difficult to manufacture.

What is needed in the art are disposable absorbent articles (e.g., a diaper) with enhanced stretch capability that are relatively inexpensive, simple to manufacture, comfortable, and convenient to use.

### SUMMARY OF THE INVENTION

The present invention provides a disposable absorbent article (e.g., diaper). The disposable absorbent article includes enhanced stretch capability that is relatively inexpensive, simple to manufacture, comfortable, and convenient to use.

Specifically, the present invention provides a disposable absorbent article comprising a stretchable multilayer chassis that defines an inner surface and an outer surface, an absorbent body having an inner surface and an outer surface wherein the stretchable multilayer is chassis stretchable in at least the cross-machine direction, and the absorbent body is affixed to the inner surface of the multilayer stretchable chassis such that in use, the inner surface of the absorbent body lies against the wearer and the stretchable multilayer chassis stretches about the wearer independently in at least the cross-machine direction.

The present invention also provides a disposable absorbent article comprising an absorbent body liner that defines an inner surface and an outer surface, an absorbent core that defines an inner surface and an outer surface, a tissue wrapsheet, a surge management layer that defines an inner surface and an outer surface, a chassis liner that defines an inner surface and an outer surface wherein the chassis liner is stretchable independently in both the cross-machine direction and the machine direction, and an outer cover that defines an inner surface and an outer surface wherein the outer cover is stretchable independently in both the cross-machine direction and the machine direction wherein the absorbent body liner houses the absorbent core, the tissue wrapsheet and the surge management layer to form an absorbent body, and the outer surface of the chassis liner is laminated to the inner surface of the outer cover forming a stretchable multilayer chassis defining an inner surface and an outer surface such that the inner surface of the absorbent core and the outer surface of the absorbent core are wrapped by a tissue wrapsheet, the inner surface of the surge management layer is adjacent to the outer surface of the absorbent body liner and the outer surface of the surge management layer is adjacent to the tissue wrapsheet which is adjacent to the inner surface of the absorbent core, and wherein the outer surface of the absorbent body liner is affixed to the inner surface of the stretchable multilayer chassis.

### BRIEF DESCRIPTION OF THE DRAWINGS

FIG. 1 illustrates a specific diaper configuration of the present invention.

FIG. 2 illustrates a cross-sectional view of a specific diaper configuration of the present invention.

FIG. 3 illustrates a typical diaper assembly.

FIG. 4 illustrates a cross sectional view of a typical diaper assembly.

### DESCRIPTION OF PREFERRED EMBODIMENTS

In the following detailed description of the preferred embodiments, reference is made to the accompanying drawing that forms a part hereof, and in which is shown by way of illustration specific embodiments in which the invention may be practiced. It is understood that other embodiments may be utilized and structural changes may be made without departing from the scope of the present invention.

The leading digit(s) of reference numbers appearing in the Figures generally correspond to the Figure number in which that component is first introduced, such that the same reference number is used throughout to refer to an identical component which appears in multiple Figures. Signals and connections may be referred to by the same reference number or label, and the actual meaning will be clear from its use in the context of the description.

The present invention described herein is directed to stretchable disposable absorbent articles. While the present description will particularly be made in the context of a diaper article, it should be understood that the present invention is also applicable to other disposable personal care absorbent articles, such as adult incontinence articles, sanitary napkins, children's training pants and the like.

As used herein, “disposable absorbent article” refers to a disposable article which absorbs and contains body exudates. Typically, they are intended to be discarded after a limited period of use. The articles are not intended to be laundered or otherwise restored for reuse. The articles can be placed against or in proximity to the body of the wearer to absorb and contain various exudates discharged from the body.

As used herein, “absorbent body” refers to the materials of the absorbent article that are intended to provide the primary absorbency capabilities of the article, such as the absorbent core. Materials associated with the absorbent core may also be included in the absorbent body, for example, any tissue or nonwoven layers, acquisition layers, and the like or combinations thereof may also be contemplated to be part of the absorbent body.

As used herein, “chassis” refers to the body or frame of the disposable absorbent article. It will typically include one or more layers of suitable material. In the present invention, these layers may include, but are not limited, the liner and the outer cover.

As used herein, the term “layer” when used in the singular may have the dual meaning of a single element or a plurality of elements such as film, woven, nonwoven, laminate, composite, or the like, whether pervious or impervious to air, gas, and/or liquids.

As used herein, the term “surface” refers to the inner or outer boundary of a layer.

As used herein, the term “inner” refers to a surface that faces the wearer when in use.

As used herein, the term “outer” refers to a surface opposite that which faces the wearer when in use.

As used herein, the term “leg elastic member” and “waist elastic member” refer to elastic material generally adapted to fit about the legs and waist of a wearer in use to maintain a positive, contacting relationship with the wearer to effectively reduce or eliminate the leakage of body exudates from the diaper.

As used herein, the term “liquid permeable” refers to the ability of liquid, such as urine, to readily penetrate through the thickness of a layer or laminate under ordinary use conditions in a direction generally perpendicular to the plane of the layer or laminate at the point of liquid contact.

As used herein, the term “liquid impermeable” refers to the inability of liquid, such as urine, to readily penetrate through the thickness of a layer or laminate under ordinary use conditions in a direction generally perpendicular to the plane of the layer or laminate at the point of liquid contact.

As used herein, the term “hydrophilic” describes fibers or the surfaces of fibers that are wetted by the aqueous liquids in contact with the fibers. The degree of wetting of the materials can, in turn, be described in terms of the contact angles and the surface tensions of the liquids and materials involved.

As used herein, the term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding and hydrophobic associations.

As used herein, “thermal point bonding” refers to passing a fabric or web of fibers to be bonded between a heated calender roll and an anvil roll. The calender roll is usually, though not always, patterned in some way so that the entire fabric is not bonded across its entire surface.

As used herein, “ultrasonic bonding” refers to a process performed, for example, by passing the fabric between a sonic horn and anvil roll.

As used herein, “adhesive bonding” refers to an adhesive, such as a hot melt adhesive, that is applied between a film and a non-woven fiber material to bind the film and non-woven together. The adhesive can be applied for example, by melt spraying, printing or meltblowing.

As used herein, the term “extensible” refers to that property of a material where upon removal of an extending force, it provides a substantially permanent deformation and/or does not exhibit a significant retractive force.

As used herein, the term “elastic,” or “elastomeric” refers to that property of a material where upon removal of an extending force, it is capable of substantially recovering its original size and shape and/or exhibits a significant retractive force.

As used herein, the term “stretch,” or “stretchable” refers to a material that is either elastic or extensible. That is, the material is capable of being extended, deformed, or the like, without breaking, and may or may not significantly retract after removal of an extending force.

As used herein, the term “biaxial stretch” refers to a material having stretchability in two directions perpendicular to one another, e.g. stretchability in a machine direction and in a cross machine direction, or in a longitudinal direction (front to back) and a lateral direction (side to side).

As used herein, the term “necked” or “neck-stretched” interchangeably refer to a method of elongating a nonwoven fabric, generally in the longitudinal, or machine direction, to reduce its width in a controlled manner to a desired amount. The controlled stretching may take place under cool, room temperature or greater temperatures and is limited to an increase in overall dimension in the direction being stretched up to the point required to break the fabric. When relaxed, the web retracts toward its original dimensions.

As used herein, the term “film” refers to a thermoplastic film made using a film extrusion and/or foaming process, such as a cast film or blown film extrusion process. For the purposes of the present invention, the term includes nonporous films as well as microporous films. Films may be vapor permeable or vapor impermeable, and function as liquid barriers under normal use conditions.

As used herein, the term “join” refers to the condition where a first member, or component, is directly affixed, adhered, or otherwise connected to a second member or component such as when each is directly bonded to intermediate elements.

As used herein, “affixed” or “bonded” refers to the joining, adhering, connecting, attaching, or the like, of two elements. Two elements will be considered to be bonded together when they are bonded directly to one another or indirectly to one another.

As used herein, the term “thermoplastic” refers to uncrosslinked polymers of a thermally sensitive material which flows under the application of heat or pressure.

As used herein, the term “machine direction” refers to the longitudinal direction.

As used herein, the term “cross-machine direction” refers to the lateral direction, i.e., a direction generally perpendicular to the machine direction.

As used herein the term “longitudinal” refers to the longitudinal axis that lies in the plane of the article and is generally parallel to a vertical plane that bisects a standing wearer into left and right body halves when the article is worn.

As used herein the term “transverse” refers to the axis which lies in the plane of the article generally perpendicular to the longitudinal axis. The article as illustrated is longer in the longitudinal direction than in the transverse direction.

As used herein, the term “polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

As used herein, the term “metallocene polymers” refers to those polymer materials that are produced by the polymerization of at least ethylene using metallocenes or constrained geometry catalysts, a class of organometallic complexes, as catalysts.

As used herein, the term “nonwoven” and “nonwoven web” refer to fibrous materials and webs of fibrous material which are formed without the aid of a textile weaving or knitting process.

As used herein, “bonded carded” refers to staple fibers which are usually purchased in bales. The bales are placed in a picker which separates the fibers. Next, the fibers are sent through a combing or carding unit which further breaks apart and aligns the staple fibers in the machine direction so as to form a machine direction-oriented fibrous non-woven web. Once the web has been formed, it is then bonded by one or more of several bonding method powder bonding and pattern bonding.

As used herein, “spunbonded fibers” refers to small diameter fibers which are formed by extruding molten thermoplastic material as filaments from a plurality of fine capillaries of a spinnerette having a circular or other configuration, with the diameter of the extruded filaments then being rapidly reduced.

As used herein, “meltblown fiber” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter (the average microfiber diameter is not greater than about 100 microns, for example, having an average diameter of from about 0.5 microns to about 50 microns, more particularly, microfibers may have an average diameter of from about 4 microns to about 40 microns).

As used herein, “superabsorbent” refers to a water-swellable, water-insoluble organic or inorganic material capable, under the most favorable conditions, of absorbing at least about 15 times its weight and, more desirably at least about 30 times its weight in an aqueous solution containing 0.9 weight percent sodium chloride. The superabsorbent materials can be natural, synthetic, or a combination thereof.

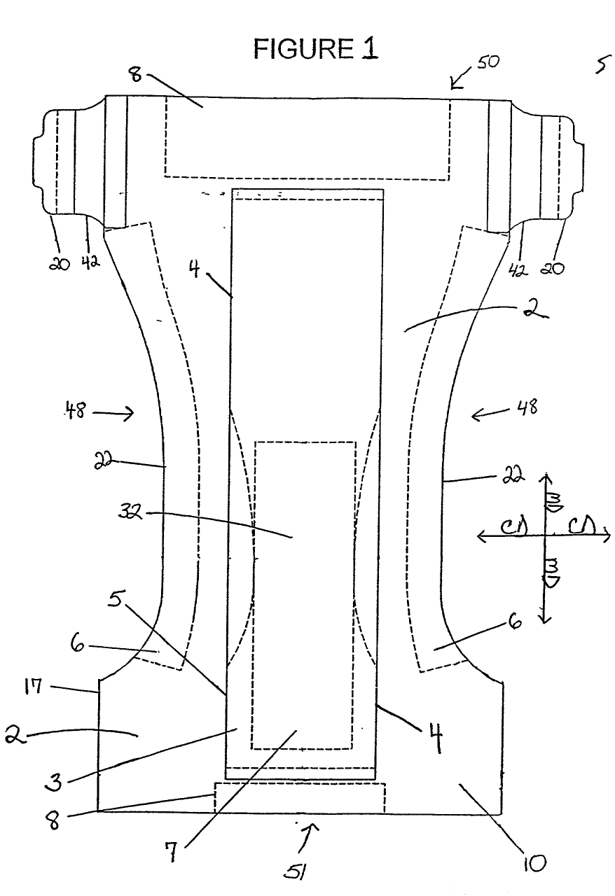

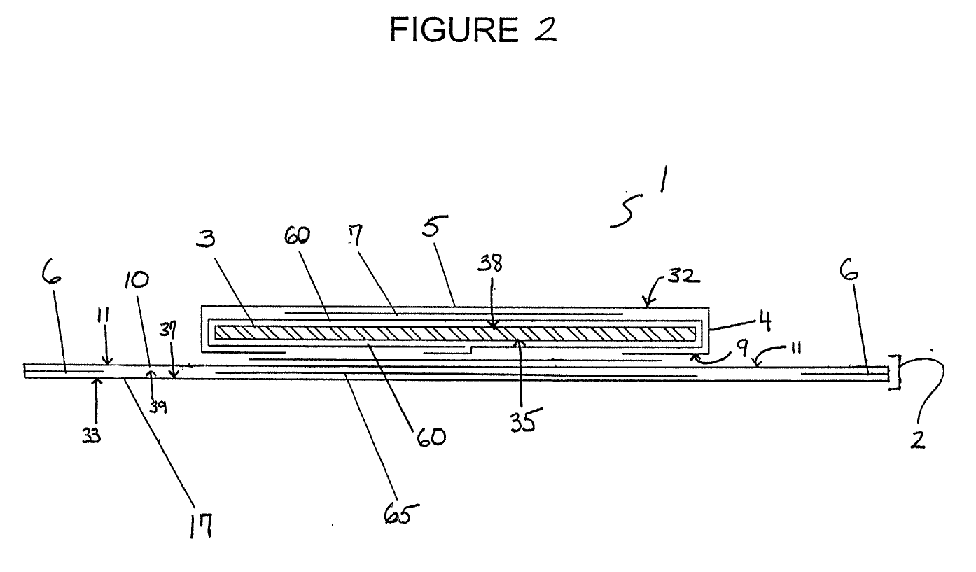

As representatively illustrated in FIG. 1 and FIG. 2, the present invention provides a diaper 1 featuring a stretchable chassis 2 and an absorbent body 4 that is affixed thereto. In particular, the absorbent body 4 is affixed to the chassis liner 10 of the stretchable chassis 2 advantageously providing a stretchable diaper 1 that provides pleasing stretch capabilities yet may be manufactured in a more efficient and cost effective manner.

The diaper 1 of the present invention is representatively illustrated in FIG. 1 as a top view which illustrates one embodiment of the diaper 1 assembly. The configuration of the diaper 1 may be of various suitable shapes. For example, in the unfastened configuration, the diaper 1 may have a generally rectangular shape, T-shape, I-shape, hourglass shape, or a combination thereof. The present embodiment illustrates the diaper 1 having a generally hourglass shape in an unfastened flat position.

The diaper 1 generally defines a front waist region 51 and a back waist region 50 which together define a three-dimensional diaper 1 configuration having a waist opening and a pair of leg openings (not shown). In use, diaper 1 is applied to the wearer by positioning the back waist region 50, around the wearer's back and drawing the remainder of the diaper 1 (i.e., the front waist region 51) between the legs of the wearer so that the front waist region 51 of the diaper 1 is disposed across the front of the wearer and the back waist region 50 of the diaper 1 is disposed across the rear of the wearer. The back waist region 50 and the front waist region 51 of the diaper 1 are configured to encircle the waist of the wearer when worn and provide a waist opening. The back waist region 50 and the front waist region 51 are interconnected by a crotch region 48. Portions of the transversely opposed side edges of the crotch region 48 generally define the leg openings.

The various components of the diaper 1 are integrally assembled together employing various types of suitable attachment means, such as with adhesives, sonic bonding, thermal bonding or a combination thereof. In particular, the diaper 1 of the present invention features a chassis 2 and an absorbent body 4. The absorbent body 4 may be connected to the chassis 2 to form the diaper 1 using means as are well known to those skilled in the art, as will be described in greater detail below.

The chassis 2 of the diaper 1 of the present invention features a stretchable outer cover 17, a stretchable chassis liner 10 as well as several components that may optionally be included in the diaper 1 to improve the overall performance of the diaper 1. For example, the chassis 2 of diaper 1 may include fastening means, such as hook and loop fasteners 20, to secure the diaper 1 on a wearer. Alternatively, other fastening means such as buttons, pins, snaps, adhesive tape fasteners, cohesives, mushroom-and-loop fasteners, or the like, may be employed. The fasteners 20 may be located at the rear waist region 50, the front waist region 51, or both the rear waist region 50 and the front waist region 51 of the diaper 1. For example, in the representatively shown embodiment, each of the fasteners 20 are assembled and attached to extend from the side panels 42 that are attached to the laterally opposed side edges in the back waist region 50 of the diaper 1. Such fastening systems generally comprise a “hook” or hook-like, male component, and a cooperating “loop” or loop-like, female component which engages and releasably interconnects with the hook component. Desirably, the interconnection is selectively releasable and re-attachable.

Conventional hook and loop fastening systems are, for example, available under the VELCRO trademark. In a particular embodiment, the fasteners 20 may be a microhook material such as that distributed under the designation CS200 by 3M Company, a business having offices in St. Paul, Minn. Another suitable micro-hook material is distributed under the designation VELCRO 851, and is available from VELCRO U.S.A., Inc., a business having offices in Manchester, N.H.

The loop element may be provided directly by the outer cover 17 of the chassis 2 to provide a “fasten anywhere” mechanical fastening system for improved fastening. Alternatively, the diaper 1 may include one or more attachment panels (not shown) to which the fasteners 20 are configured to releasably engage. For example, when the fasteners 20 are hook fasteners located in the back waist region 50 of the diaper 1 as illustrated, the diaper 1 may include a corresponding attachment panel such as a complementary loop element on the outward facing surface in the front waist region 51. The attachment panels (not shown) may be provided by a woven fabric, a nonwoven fabric, a knitted fabric, a perforated or apertured layer, and the like, as well as combinations thereof. For example, a suitable material for the attachment panel can be composed of a 2 bar, warp knit fabric of the type available from Guilford Mills, Inc., Greensboro, N.C. under the trade designation #34285, as well other of knit fabrics. Alternatively, a pattern unbonded nonwoven material may be suitably used for an attachment panel, as described in U.S. Pat. No. 5,858,515 issued Jan. 12, 1999 to Stokes, et al..

As mentioned above, the chassis 2 may also include side panels 42. The side panels 42 may be an elastomeric material such as a neck-bonded laminate (NBL) or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al., the disclosures of which are hereby incorporated by reference. Examples of articles that include elasticized side panels and selectively configured fastener tabs are described in U.S. Pat. No. 5,496,298 issued Mar. 5, 1996 to Kuepper et al.; U.S. Pat. No. 5,540,796 to Fries; and U.S. Pat. No. 5,595,618 to Fries; the disclosures of which are also incorporated herein by reference.

The chassis 2 may further include a ventilation layer 65 (See FIG. 2) located between the chassis liner 10 and the outer cover 17 to insulate the outer cover 17 from the absorbent body 4 and to reduce the dampness of the outer surface 33 of the outer cover 17.

The chassis 2 of the diaper 1 of the present invention may also include a pair of containment flaps (not shown) that are configured to provide a barrier and to contain the lateral flow of body exudates. The containment flaps may be generally located along the laterally opposed side edges 22 of the diaper 1 and generally adjacent the side edges of the absorbent body 4. The containment flaps are located on the side edges of the absorbent body liner 5. Each containment flap typically defines an unattached edge which is configured to form a seal against the wearer's body. The containment flaps may extend longitudinally along the entire length of the absorbent body 4 or may only extend partially along the length of the absorbent body 4. When the containment flaps are shorter in length than the absorbent body 4, the containment flaps may be selectively positioned anywhere along the absorbent body 4 in the longitudinal direction (i.e., the machine direction). In a particular aspect of the invention, the containment flaps extend along the entire length of the absorbent body 4 in the longitudinal direction to better contain the body exudates. Such containment flaps are generally well known to those skilled in the art. For example, suitable constructions and arrangements for containment flaps are described in U.S. Pat. No. 4,704,116 issued Nov. 3, 1987, to K. Enloe, the disclosure of which is hereby incorporated by reference.

Other diaper components, such as the leg elastic members 6, the waist elastic members 8 and the fasteners 20, may be assembled into the chassis 2 by using means as are well known to those skilled in the art. For example, the fasteners 20 may be connected to the outer cover 17 with an adhesive. The adhesive may be applied as a uniform continuous layer of adhesive, a patterned layer of adhesive, a sprayed pattern of adhesive, or any of separate lines, swirls or dots of adhesive. Alternatively, the fasteners 20 may be attached to the outer cover 17 by ultra sonic bonding, thermal bonding, or the like.

Materials suitable for use as the leg elastic members 6 and waist elastic members 8 are also well known to those skilled in the art. Exemplary of such materials are sheets or strands or ribbons of a polymeric, elastomeric material which may be adhered to the outer cover 17 in a stretched position, or which may be attached to the outer cover 17 while the outer cover is pleated, such that elastic constrictive forces are imparted to the outer cover 17. The leg elastic members 6 may also include such materials as polyurethane, synthetic rubber, natural rubber, or a combination thereof.

Diaper components such as the leg and waist elastic members 6 and 8 may be interposed between the outer cover 17 and the chassis liner 10 and may be affixed to the inner surface 37 of the outer cover 17, the outer surface 39 of the chassis liner 10, or both (See FIG. 2). The fasteners 20 may be affixed to the inner surface 11 of the chassis 2, the outer surface 39 of the chassis liner 10, the inner surface 37 of the outer cover 17, or the outer surface 33 of the chassis 2. Alternately, components such as the leg and waist elastic members 6 and 8 may be affixed to the inner surface 11 of the chassis liner 10.

Chassis Liner

In the preferred embodiment of the present invention, the stretchable chassis liner 10 is extensible. The chassis liner 10 may be extensible independently in the cross-machine direction, the machine direction, or both the cross machine direction and the machine direction. The chassis liner 10 of the present invention is preferably extensible in at least the cross-machine direction. The stretchable chassis liner 10 includes extensible properties that are capable of stretching with the outer cover 17 and permanently and independently deforming in at least the lateral direction (i.e., cross-machine direction).

The chassis liner 10 desirably has the same dimensions as the stretchable outer cover 17 and is laminated to the outer cover 17 allowing the chassis liner 10 to stretch with the outer cover 17. The stretch of the diaper 1 is desirable to provide improved fit for the wearer. For example, as the diaper 1 is applied to the wearer, the caregiver typically stretches the diaper 1 around the waist and buttocks of the wearer creating tension forces in the chassis 2 in the transverse, or cross-machine direction. Further, waist circumference and buttock variations due to the movement of the wearer, such as the bending and breathing of the wearer as well as the discharge of body exudates, create transverse stresses in the chassis 2. In addition, as the back waist region 50 and the front waist region 51 of the diaper 1 are fitted about the wearer, tension forces are created in the longitudinal, or machine direction in the crotch region and the front torso region of the diaper 1. The stretch of the chassis 2 provides the necessary stretch and, in certain embodiments, retraction to allow the diaper 1 to maintain a high level of fit as the dimensions of the wearer's waist, buttocks and crotch change.

Portions of the inner surface 11 of the stretchable chassis 2 are desirably in contact with the wearer's skin as the chassis 2 contains a greater surface area than the surface area of the absorbent body 4 to encompass the back waist region 50 and the front waist region 51 of the wearer. The stretchable inner surface 11 of the chassis liner 10 desirably provides a generally soft cloth-like texture on at least those portions of the inner surface 11 that rests against the wearer's skin. One example of such a generally cloth-like material is a thermoplastic nonwoven web, such as a spunbond thermoplastic nonwoven web made from a stretchable polymer.

The chassis liner 10 may be laminated to the outer cover 17 forming the chassis 2 using means as are well known to those skilled in the art such as utilizing adhesives, ultra sonic bonding, thermal bonding, or the like.

A suitable stretchable chassis liner 10 may be manufactured from a wide selection of web materials, such as porous foams, reticulated foams, apertured plastic films, natural fibers (for example, wood or cotton fibers), synthetic fibers (for example, polyester or polypropylene fibers), or a combination thereof.

The stretchable chassis liner 10 may be composed of various extensible materials such as a necked fabric, a creped fabric, a micro-pleated fabric, perforated polymer films, or the like, or a combination thereof. The fabrics may be non-elastic woven or nonwoven materials, such as spunbond fabrics. Examples of suitable manufacturing techniques and suitable necked nonwoven fabric materials for such an extensible chassis liner 10 are described in U.S. Pat. No. 4,965,122 entitled REVERSIBLY NECKED MATERIAL, by M. T. Morman which issued Oct. 23, 1990.

The stretchable chassis liner 10 may be made from non-elastic neckable materials for reduced cost and improved manufacturing efficiency. Suitable non-elastic neckable materials include nonwoven webs, woven materials and knitted materials. Such webs can include one or more fabric layers. Nonwoven fabrics or webs have been formed from many processes, for example, bonded carded web processes, meltblowing processes and spunbonding processes. The non-elastic neckable material is preferably formed from at least one member selected from fibers and filaments of inelastic polymers. The polymers include polyesters, for example, polyethylene terephthalate, polyolefins, for example, polyethylene and polypropylene, polyamides, for example, nylon 6 and nylon 66. These fibers or filaments are used alone or in a mixture of two or more thereof.

Suitable fibers for forming the neckable material include natural and synthetic fibers as well as bicomponent, multi-component, and shaped polymer fibers. Many polyolefins are available for fiber production according to the present invention, for example, fiber forming polypropylenes include Exxon Chemical Company's Esbodyne® PD 3445 polypropylene and Himont Chemical Company's PF-304. Polyethylenes such as Dow Chemical's ASPUN® 6811A linear low density polyethylene, 2553 LLDPE and 25355 and 12350 high density polyethylene are also suitable polymers. The nonwoven web layer may be bonded to impart a discrete bond pattern with a prescribed bond surface area. If too much bond area is present on the neckable material, the material will break before it necks. If there is not enough bond area, then the neckable material will pull apart. Typically, the percent bonding area useful in the present invention ranges from around 5 percent to around 40 percent of the area of the neckable material.

For example, a particularly suitable stretchable material for the chassis liner 10 is a necked spunbond web of polypropylene fibers having a basis weight of from about 5 to about 30 gsm before necking. Such a web may be necked up to about 80 percent. The neckable material may be necked to form the stretchable chassis liner 10 by conventional necking processes that typically vary the surface speed of the web to draw or neck the material. Such necking will allow the material to stretch in the transverse direction. The necked nonwoven fabric materials are typically capable of being necked up to about 80 percent (i.e., narrowed to about 20 percent of its original width). For example, the extensible chassis liner 10 of the various aspects of the present invention may be necked from about 10 to about 80 percent, desirably from about 20 to about 60 percent, and more desirably from about 30 to about 50 percent for improved performance.

When provided by extensible materials as described above, the stretchable chassis liner 10 may provide a substantially permanent deformation of at least about 10 percent, desirably at least about 20 percent, and more desirably at least about 30 percent when subjected to a tensile force of 100 gmf per inch (per 2.54 cm) of width of the test sample according to the Material Elongation and Deformation Tensile Test set forth herein. This permanent deformation causes the material to pucker up between material/elastomer attachment points when the elastomer that it is attached to retracts. Substantially permanent deformations less than those set forth above may not provide the desired permanent deformation for improved fastening, softness, containment and fit.

In still other aspects, the chassis liner 10 may provide a substantially permanent deformation of from about 10 to about 100 percent and desirably from about 20 to about 80 percent when subjected to the tensile force of 100 gmf per inch (per 2.54 cm) of width of the test sample according to the Material Elongation and Deformation Tensile Test set forth herein. The stretchable chassis liner 10 may also provide an elongation of at least about 20 percent, desirably at least about 25 percent and more desirably at least about 30 percent when subjected to a tensile force of 100 gmf per inch (per 2.54 cm) of width of the test sample according to the Material Elongation and Deformation Tensile Test set forth herein for improved performance.

In an alternate embodiment of the present invention, the stretchable chassis liner 10 is elastic. The stretchable chassis liner 10 may be stretchable, independently in the cross-machine direction, the machine direction, or both the cross machine direction and the machine direction. In this embodiment, the chassis liner 10 is preferably elastic independently in both the cross-machine direction and the machine direction. In such a configuration, the chassis liner 10 desirably has the same dimensions as the stretchable outer cover 17 and the same biaxial stretch properties as the outer cover 17 to enable the chassis liner 10 to stretch and retract with the biaxially stretchable outer cover 17 for improved fastening, softness, fit and containment of body fluids.

The stretchable chassis liner 10 may suitably be composed of a neck-stretched, spunbond web with KRATON® G strands, such as 0.4 osy (60% neck-stretched) polypropylene spunbond laminated to 0.4 osy strands of KRATON® MM G2760 with 12 strands per inch, which is stretched perpendicular to the necking direction, then allowed to retract. Alternately, the stretchable chassis liner 10 may include a KRATON® Film.

The stretchable chassis liner 10 may be composed of a substantially hydrophobic material, and the hydrophobic material can, optionally, be treated with a surfactant or otherwise processed to impart a desired level of wettability and hydrophilicity. For example, the elastic material can be surface treated with about 0.45 weight percent of a surfactant mixture including AHCOVEL® N-62 from Hodgson Textile Chemicals of Mount Holly, N.C., U.S.A. and GLUCOPON® 220UP from Henkel Corporation of Ambler, Pa., in an active ratio of 3:1. The surfactant can be applied by any conventional means, such as spraying, printing, brush coating or the like. The surfactant can be applied to the entire stretchable chassis liner 10 or can be selectively applied to particular sections of the stretchable chassis liner 10, such as the medial section along the longitudinal centerline.

Outer Cover

The outer cover 17 of the present invention is desirably comprised of elastic materials. The stretchable outer cover 17 may be stretchable independently in the cross-machine direction, the machine direction, or both the cross machine direction and the machine direction. The outer cover 17 of the present invention is preferably elastic in at least the cross-machine direction. Elastic materials allow the outer cover to stretch and retract biaxially under the stress tensions created by the movement of the wearer. The improved fit and aesthetics of the diaper 1 is due to the stretchable and retractable characteristics of the outer cover 17 of the chassis 2 when lateral and longitudinal stress forces are imposed on the stretchable chassis 2 over the buttock and waist areas of the wearer.

Moreover, the enhanced stretch of a biaxial elastic outer cover 17 improves diaper 1 containment. As the absorbent core 3 absorbs fluid exudates and expands outwardly, the chassis 2 may readily stretch in correspondence with the expansion of the absorbent core 3 and/or other absorbent components of the diaper 1 such as the tissue wrapsheet 60, the surge management layer 7, etc. to more effectively contain the exudates. For example, as the absorbent core 3 is insulted by urine, the urine may be distributed by a wicking layer such as the surge management layer 7 toward the longitudinal ends of the absorbent core 3 as well as the transverse edges of the absorbent core 3. The distribution of urine to the various portions of the absorbent core 3 may create outward longitudinal and transverse tensions on the outer cover 17 (and the chassis liner 10). The ability of a stretchable chassis 2 of the present invention to stretch and retract with these longitudinal and transverse tensions improves the fit, comfort and appearance of the diaper 1 on the wearer.

One example of an elastic outer cover 17 material with elastic properties is a 0.3 osy polypropylene spunbond that is necked 60% in the transverse direction (i.e., cross-machine direction) and creped 60% in the longitudinal direction, laminated with 3 grams per square meter (gsm) Findley 2525A styrene-isoprene-styrene based adhesive to 8 gsm PEBAX® 2533 film with 20% TiO2 concentrate. In such an elastic embodiment, the outer cover 17 may suitably be stretched, transversely and/or longitudinally, by at least 25% (to at least 125% of an initial (unstretched) width and/or length of the outer cover 17). More suitably, the outer cover 17 may be stretched, transversely and/or longitudinally, by at least 50% (to at least 150% of an initial (unstretched) width and/or length of the outer cover 17). Even more suitably, the outer cover 17 may be stretched, transversely and/or longitudinally, by at least 100% (to at least 200% of the unstretched width or length of the outer cover 17). Most more suitably, the outer cover 17 may be stretched, transversely and/or longitudinally, by at least 150% (to at least 250% of the unstretched width or length of the outer cover 17). Tension in the outer cover 17 at 50% extension is suitably between 50 and 1000 grams, more suitably between 100 and 600 grams, as measured on a 3 inch wide piece of the outer cover material.

A stretchable outer cover 17 desirably includes elastic material that is substantially liquid impermeable. The stretchable outer cover 17 may be a single layer of liquid impermeable material, or may also include a multi-layered laminate structure in which at least one of the layers is liquid impermeable. For example, the stretchable outer cover 17 may include a liquid permeable outer layer and a liquid impermeable inner layer that are suitably joined together by a laminate adhesive or thermal bonded attachment means. Suitable laminate adhesives, which can be applied continuously or intermittently as beads, a spray, parallel swirls, or the like, may be obtained from Bostik-Findley Adhesives, Inc., of Wauwatosa, Wis., U.S.A., or from National Starch and Chemical Company, Bridgewater, N.J., U.S.A.

The liquid permeable outer layer may be any suitable material and desirably one that provides a generally cloth-like texture. One example of such a material is a thermoplastic nonwoven web, such as a spunbond thermoplastic nonwoven web made from a stretchable polymer and having a basis weight of about 1-100 grams per square meter (gsm), suitably about 5-50 gsm, more suitably 10-30 gsm. Suitable stretchable polymers for making the nonwoven web include certain flexible polyolefins, for example propylene-based polymers having both atactic and isotactic propylene groups in the main polypropylene chain. Also included are heterophasic propylene-ethylene copolymers. Heterophasic polymers are reactor blends formed by adding different levels of propylene and ethylene at different stages in the reactor. Heterophasic polymers typically include about 10-90% by weight of a first polymer segment A, about 10-90% by weight of a second polymer segment B, and 0-20% by weight of a third polymer segment C. Polymer segment A is at least about 80% crystalline and includes about 90-100% by weight propylene, as a homopolymer or random copolymer with up to 10% by weight ethylene. Polymer segment B is less than about 50% crystalline, and includes about 30-70% by weight propylene randomly copolymerized with about 30-70% by weight ethylene. Optional polymer segment C contains about 80-100% by weight ethylene and 0-20% of randomly copolymerized propylene.

Other stretchable polymers include very low density polyethylene (VLDPE), which is an ethylene-alpha olefin copolymer having a density less than 0.900 grams/cm3, preferably about 0.870-0.890 grams/cm3. Preferred VLDPE's are single-site catalyzed. Other stretchable polymers include random propylene-alpha olefin copolymers containing more than 10% by weight of a C2 or C4-C12 comonomer, preferably about 15-85% by weight of the comonomer, with ethylene being a preferred comonomer.

The inner layer of the stretchable outer cover 17 is desirably manufactured from a thin (1-50 microns, suitably 5-25 microns, more suitably 10-20 microns) plastic film, although other stretchable liquid impermeable materials may also be used. The film layer of the outer cover 17 may contain a blend of a thermoplastic polymer and a 30-70% by weight of a particulate inorganic filler, such as calcium carbonate. The film can be oriented at least uniaxially to cause void formation around the filler particles, resulting in breathability. It should be generally understood that such a liquid impermeable film alone could suitably provide the outer cover 17.

Suitable stretchable polymers for making the film include stretchable olefin polymers, such as an olefinic copolymer of polyethylene. More specifically, other stretchable polymers include diblock, triblock, tetrablock or other multi-block elastomeric copolymers such as olefinic copolymers, including styrene-isoprene-styrene, styrene-butadiene-styrene, styrene-ethylene/butylene-styrene, or styrene-ethylene/propylene-styrene, which may be obtained from the Shell Chemical Company, under the trademark KRATON® elastomeric resin; polyurethanes, including those available from E. I. du Pont de Nemours Co., under the trademark LYCRAV® polyurethane; polyamides, including polyether block amides available from Ato Chemical Company, under the trademark PEBAX® polyether block amide; polyesters, such as those available from E. I. Du Pont de Nemours Co., under the trademark HYTREL® polyester; and single-site or metallocene-catalyzed polyolefins having density less than about 0.91 grams/cc, available from Dow Chemical Co. under the trademark AFFINITY®.

Other suitable materials for the elastic outer cover 17 may include a spunbonded laminate, a meltblown laminate, a spunbond-meltblown-spunbond laminate, or a stretch-bonded laminate (SBL) made using a stretchable polymer or blend thereof. A more specific example of suitable liquid impermeable films for use as a liquid impermeable inner layer, or as a single layer liquid impermeable stretchable outer cover 17, is a 0.02 millimeter polyethylene film commercially available from Pliant Corp. Newport News, Va., U.S.A. If the stretchable outer cover 17 is a single layer of material, it can be embossed and/or matte finished to provide a more cloth-like appearance. The liquid impermeable material can permit vapors to escape from the interior of the disposable absorbent article, while still preventing liquids from passing through the stretchable outer cover 17.

Alternatively, the stretchable outer cover 17 may be extensible independently in the cross-machine direction, the machine direction, or both the cross-machine direction and the machine direction. In the alternative embodiment, the stretchable outer cover 17 is extensible independently in both the cross-machine direction and the machine direction. When provided by the extensible materials as described below, the outer cover 17 of the present invention is desirably capable of providing a selected elongation when subjected to an applied tensile force and capable of providing a selected, sustained deformation when subjected to an applied tensile force and then allowed to relax for a selected time period after removing the applied tensile force. The measurement of the selected time period begins immediately after the removal of the tensile force. The selected elongation and sustained deformation occurs at least along the lateral direction (i.e., cross-machine direction) and longitudinal (i.e., machine direction) of the diaper 1.

In particular aspects where the stretchable outer cover 17 is extensible, the outer cover 17 may provide an elongation of at least about 10 percent, desirably at least about 20 percent, more desirably at least about 30 percent and even more desirably at least about 40 percent when subjected to a tensile force of 100 gmf per inch (per 2.54 cm) of width of the test sample according to the Material Elongation and Deformation Tensile Test set forth herein. Elongation less than those above may not provide the desired stretch for improved fastening, containment and fit. In other aspects, the stretchable outer cover 17 may be capable of providing an elongation of from about 10 percent to about 200 percent and desirably from about 30 percent to about 100 percent when subjected to a tensile force of 100 gmf per inch (per 2.54 cm) of width of the test sample according to the Material Elongation and Deformation Tensile Test set forth herein.

In certain aspects, the stretchable outer cover 17 may also provide a substantially permanent deformation of at least about 10 percent, desirably at least about 15 percent, more desirably at least about 17 percent, even more desirably at least about 20 percent, further more desirably at least about 25 percent and even further more desirably at least about 30 percent when subjected to a tensile force of 100 gmf per inch (per 2.54 cm) of width of the test sample according to the Material Elongation and Deformation Tensile Test set forth herein. Substantially permanent deformations less than those set forth above may not provide the desired improved fastening, containment, enhanced buttock coverage and fit. In still other aspects, the stretchable outer cover 17 may provide a substantially permanent deformation of from about 10 to about 100 percent and desirably from about 17 to about 100 percent when subjected to the tensile force of 100 gmf per inch (per 2.54 cm) of width of the test sample according to the Material Elongation and Deformation Tensile Test set forth herein. It should be noted that the permanent deformation properties of the extensible outer cover 17 are determined when the outer cover 17 is dry.

An extensible outer cover 17 may include necked fabrics, creped fabrics, crimped fiber fabrics, extendable fiber fabrics, bonded-carded fabrics, micro-pleated fabrics, polymer films, or a combination thereof. The fabrics may be woven or nonwoven materials, such as spunbond fabrics. In a particular embodiment, the stretchable outer cover 17 may be composed of a extensible laminate of two or more necked layers.

Typically, a necked nonwoven fabric material is capable of being necked up to about 80 percent. For example, the stretchable outer cover 17 of the various aspects of the present invention may be provided by a material that has been necked from about 10 to about 80 percent, desirably from about 20 to about 60 percent, and more desirably from about 30 to about 50 percent for improved performance.

In a particular embodiment, the stretchable outer cover 17 may be made from a necked laminate material to provide the desired levels of stretch as well as liquid impermeability and vapor permeability. For example, the stretchable outer cover 17 may be a necked laminate formed from sheet layers of at least one neckable fabric laminated to at least one film material wherein the necked laminate is biaxially stretchable in both the cross-machine direction and the cross machine direction and does not appreciably retract. Suitable necked laminates that include at least one non-elastic neckable material laminated to at least one non-elastic film material are described in U.S. patent application Ser. No. 09/455,513 filed Dec. 6, 1999 and entitled “TRANSVERSELY EXTENSIBLE AND RETRACTABLE NECKED LAMINATE OF NON-ELASTIC SHEET LAYERS”, the entire disclosure of which is hereby incorporated by reference.

The non-elastic film layer may be made from either cast or blown film equipment and may be coextruded and can be embossed if so desired. The film layer may be made from any suitable non-elastic polymer composition and may include multiple layers. The non-elastic film layer may also be breathable. For example, the non-elastic film layer may contain such fillers as micropore developing fillers, e.g. calcium carbonate; opacifying agents, e.g. titanium dioxide; and antiblock additives, e.g. diatomaceous earth. Suitable polymers for the non-elastic film layer include, but are not limited to, non-elastic extrudable polymers such as polyolefin or a blend of polyolefins, nylon, polyester, ethylene vinyl alcohol, or a combination thereof. More particularly, useful polyolefins include polypropylene and polyethylene. Other useful polymers include those described in U.S. Pat. No. 4,777,073 to Sheth, assigned to Exxon Chemical Patents Inc., such as a copolymer of polypropylene and low density polyethylene or linear low density polyethylene.

Alternative polymers for the film layer include those referred to as single site catalyzed polymers such as “metallocene” polymers produced according to a metallocene process and which have limited elastic properties. For example, a common metallocene is ferrocene, a complex of a metal between two cyclopentadienyl (Cp) ligands. Such metallocene polymers are available from Exxon Chemical Company of Baytown, Tex. under the trademark EXXPOL® for polypropylene based polymers and EXACT® for polyethylene based polymers and from Dow Chemical Company of Midland, Mich. under the name ENGAGE®. Preferably, the metallocene polymers are selected from copolymers of ethylene and 1-butane, copolymers of ethylene and 1-hexene, copolymers of ethylene and 1-octene, or a combination thereof. Suitable non-elastic neckable materials for the outer cover 17 include nonwoven webs, woven materials, knitted materials, or a combination thereof, such as those described in the above-mentioned U.S. Pat. No. 4,965,122.

Nonwoven fabrics or webs have been formed from many processes, for example, bonded carded web processes, meltblowing processes and spunbonding processes. The non-elastic neckable material is preferably formed from at least one member selected from fibers and filaments of inelastic polymers. Such polymers include polyesters, for example, polyethylene terephthalate, polyolefins, for example, polyethylene and polypropylene, polyamides, for example, nylon 6 and nylon 66. These fibers or filaments are used alone or in a mixture of two or more thereof. Suitable fibers for forming the neckable material include natural fibers, synthetic fibers, bicomponent fibers, multi-component fibers, shaped polymer fibers, or a combination thereof. Many polyolefins are available for fiber production according to the present invention, for example, fiber forming polypropylenes include Exxon Chemical Company's Esbodyne® PD 3445 polypropylene and Himont Chemical Company's PF-304. Polyethylenes such as Dow Chemical's ASPUN® 6811A linear low density polyethylene, 2553 LLDPE and 25355 and 12350 high density polyethylene are also suitable polymers. The nonwoven web layer may be bonded to impart a discrete bond pattern with a prescribed bond surface area. If too much bond area is present on the neckable material, it will break before it necks. If there is not enough bond area, then the neckable material will pull apart. Typically, the percent bonding area useful in the present invention ranges from around 5 percent to around 40 percent of the area of the neckable material.

The non-elastic film layer may be laminated to the neckable material to form the laminate by conventional methods known in the art including adhesive bonding, point bonding, thermal point bonding, and sonic welding. The laminate is then necked by conventional necking processes that typically vary the surface speed of the web to draw and neck the laminate. Such necking provides striated rugosities in the film and/or laminate resulting in transverse extensibility and retractability to the necked laminate and more “cloth-like” aesthetics. It is known that stretching and orienting a filled film layer causes micropores to form in the film, but longitudinal striated rugosities do not typically form in the film layer when stretched. The film layer would instead become physically thinner and may narrow slightly. By necking the laminate, the non-elastic neckable material, which is attached to the non-elastic film layer, will neck and bring the nonelastic film layer with it, thereby forming the longitudinal striated rugosities in the film which allows the film layer to extend in the transverse direction. Alternative necked laminate materials that may be used in the outer cover 17 of the present invention are described in U.S. patent application Ser. No. 09/460,490 filed Dec. 14, 1999 and entitled “BREATHABLE LAMINATE PERMANENTLY CONFORMABLE TO THE CONTOURS OF A WEARER”, the entire disclosure of which is hereby incorporated by reference.

Accordingly, the stretchable outer cover 17 of the present invention is capable of stretching with the stretchable chassis liner 10 to provide improved fastening, softness, fit and containment of body fluids for the wearer. In particular, as the diaper 1 is applied to the wearer, the caregiver typically stretches the diaper 1 around the waist and buttocks of the wearer creating tension forces in the chassis 2 in the transverse, or cross-machine direction. Further, waist circumference and buttock variations due to the bending and breathing of the wearer also create transverse stresses in the chassis 2. In addition, as the back waist region 50 and the front waist region 51 of the diaper 1 are fitted about the wearer, tension forces are created in the longitudinal, or machine direction in the crotch region and the front torso region of the diaper 1. The biaxial stretch of the chassis 2 provides the necessary stretch and retraction to allow the diaper 1 to maintain a high level of fit as the dimensions of the wearer's waist, buttocks and crotch change.

In an alternative embodiment of the present invention, one layer of the stretchable chassis 2 may be extensible in at least the cross-machine direction and another layer of the stretchable chassis 2 may be elastic in at least the cross-machine direction such that the chassis 2 maintains elastic properties. For example, the chassis liner 10 of the chassis 2 may be extensible in at least the cross-machine direction and the outer cover 17 of the chassis 2 may be elastic in at least the cross-machine direction. A chassis 2 with the described extensible chassis liner 10 and elastic outer cover 17 combination may maintain its elastic properties as the extensible chassis liner 10 and the outer cover 17 are capable of uniformly stretching in at least the cross-machine direction.

Absorbent Body

As mentioned above, the diaper 1 of the present invention also includes an absorbent body 4. As representatively illustrated in FIGS. 1 and 2, the absorbent body 4 includes, but is not limited to an absorbent core 3, tissue wrapsheet 60, surge management layer 7, and an absorbent body liner 5.

The absorbent core 3 of the present invention may include non-stretch materials, low stretch materials, or a combination thereof. The absorbent core 3 of the diaper 1, is desirably non-extensible and non-elastic with a non-stretch capability. The absorbent core 3 of the diaper 1 may be manufactured in a wide variety of sizes and shapes (for example, rectangular, trapezoidal, T-shape, I-shape, hourglass shape, etc. or a combination thereof) and from a wide variety of materials. The absorbent core 3 may be constructed from a matrix of hydrophilic fibers, such as a web of cellulosic fluff, mixed with particles of a high-absorbency material commonly known as superabsorbent material. The absorbent core 3 may include a matrix of cellulosic fluff such as wood pulp fluff and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. The fluff and superabsorbent particles may also be selectively placed into desired zones of the absorbent core 3 to better contain and absorb body exudates. The concentration of the superabsorbent particles may also vary throughout the thickness of the absorbent core 3. Alternatively, the absorbent core 3 may include a laminate of fibrous webs and superabsorbent materials or other suitable means of maintaining a superabsorbent material in a localized area.

The high-absorbency material can be selected from natural, synthetic, modified natural polymers and materials, or a combination thereof. The high absorbency materials can be inorganic materials, such as silica gels, or organic compounds, such as crosslinked polymers. Examples of synthetic, polymeric, high-absorbency materials include the alkali metal and ammonium salts of poly(acrylic acid), poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core 3 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and the like. Mixtures of natural and wholly or partially synthetic absorbent polymers can also be useful in the present invention. Such high-absorbency materials are well known to those skilled in the art and are widely commercially available. Examples of superabsorbent polymers suitable for use in the present invention are SANWET IM 3900 polymer available from Hoechst Celanese located in Portsmouth, Va., DOW DRYTECH 2035LD polymer available from Dow Chemical Co. located in Midland, Mich. and Stockhausen W65431 polymer available from Stockhausen Inc., located in Greensboro, N.C. The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. As a general rule, the high absorbency material is present in the absorbent core 3 in an amount of from about 5 to about 90 weight percent based on total weight of the absorbent core 3.

The absorbent body 4 may also include a tissue wrapsheet 60 that encompasses the absorbent core 3 such that the oppositely facing surfaces of the absorbent core 3 are contained by the tissue wrapsheet 60. As such, the absorbent core 3 may be sealed within a tissue wrapsheet 60. A typical tissue wrapsheet 60 is a substantially hydrophilic, single-ply, low porosity creped wadding or the like. An exemplary tissue wrapsheet 60 has a basis weight of 12.5 pounds/ream, a porosity of approximately 90 cubic feet per minute per foot squared, and strength of about 500 grams.

A tissue wrapsheet 60 may be employed to help maintain the integrity of the fibrous structure of the absorbent core 3. The tissue wrapsheet 60 may also be configured to provide a wicking layer which helps to rapidly distribute liquid over the mass of absorbent fibers comprising the absorbent core. The tissue wrapsheet 60 material may be bonded to the inner surface 38 of the absorbent core 3, the outer surface 35 of the absorbent core 3, or both the inner surface 38 and the outer surface 35 of the absorbent core 3.

Further, the absorbent body 4 may include an absorbent body liner 5 that provides a pleasing surface to the wearer in use. The absorbent body liner 5 of the present invention is comprised of non-stretch materials, low stretch materials, or a combination thereof. Non-stretch and/or low stretch materials are easier and more cost effective to manufacture than extensible or elastic materials. The absorbent body liner 5 of the present invention is desirably non-extensible and non-elastic with a non-stretch capability for cost savings and ease of manufacture.

The absorbent body liner 5 suitably presents a bodyfacing surface which is compliant, soft feeling, and nonirritating to the wearers skin. The absorbent body liner 5 is suitably employed to help isolate the wearer's skin from liquids held in the absorbent core 3. The absorbent body liner 5 may be less hydrophilic than the absorbent core 3 to present a relatively dry surface to the wearer, and may be sufficiently porous to be liquid permeable, permitting liquid to readily penetrate through its thickness in order to isolate the wearer's skin from liquids held in the absorbent core 3. A suitable absorbent body liner 5 may be manufactured from a wide selection of web materials, such as porous foams, reticulated foams, apertured plastic films, natural fibers (for example, wood or cotton fibers), synthetic fibers (for example, polyester or polypropylene fibers), or a combination thereof.

Various woven and nonwoven fabrics can be used for the absorbent body liner 5. For example, the absorbent body liner 5 may be composed of a meltblown or spunbonded web of polyolefin fibers. The absorbent body liner 5 may also be a bonded-carded web composed of natural and/or synthetic fibers. The absorbent body liner 5 may be composed of a substantially hydrophobic material, and the hydrophobic material may optionally be treated with a surfactant or otherwise processed to impart a desired level of wettability and hydrophilicity. In a particular embodiment of the present invention, the absorbent body liner 5 comprises a nonwoven, spunbond, polypropylene fabric composed of about 2.8-3.2 denier fibers formed into a web having a basis weight of about 20 grams per square meter and a density of about 0.13 grams per cubic centimeter. The fabric may be surface treated with about 0.3 weight percent of a surfactant commercially available from Hodgson Textile Chemicals, Inc. under the trade designation AHCOVEL Base N-62. The surfactant may be applied by any conventional means, such as spraying, printing, brush coating or the like. The surfactant may be applied to the entire absorbent body liner 5 or may be selectively applied to particular sections of the absorbent body liner 5 to provide greater wettability of such sections. The absorbent body liner 5 may further include a composition applied thereto that is configured to be transferred to the wearer's skin for improving the skin health of the wearer. Suitable compositions for use on the absorbent body liner 5 are described in U.S. Pat. No. 6,149,934 issued Nov. 21, 2000 to Krzysik et al., the disclosure of which is hereby incorporated by reference.

As representatively illustrated in FIGS. 1 and 2, the absorbent body 4 may also include a surge management layer 7 which helps to decelerate and diffuse surges or gushes of liquid that may be rapidly introduced into the absorbent core 3 of the diaper 1. Desirably, the surge management layer 7 can rapidly accept and temporarily hold the liquid prior to releasing the liquid into the storage or retention portions of the absorbent core 3. The surge management layer 7 is preferably disposed between the absorbent body liner 5 and the absorbent core 3, but may alternatively be located on the inner surface 32 of the absorbent body liner 5 close to the wearer's skin. In the illustrated embodiment, for example, the surge management layer 7 is interposed between the absorbent body liner 5 and the absorbent core 3. Examples of suitable surge management layers 7 are described in U.S. Pat. No. 5,486,166 entitled FIBROUS NONWOVEN WEB SURGE LAYER FOR PERSONAL CARE ABSORBENT ARTICLES AND THE LIKE by C. Ellis and D. Bishop, which issued Jan. 23, 1996 and U.S. Pat. No. 5,490,846 entitled IMPROVED SURGE MANAGEMENT FIBROUS NONWOVEN WEB FOR PERSONAL CARE ABSORBENT ARTICLES AND THE LIKE by C. Ellis and R. Everett, which issued Feb. 13, 1996; the entire disclosures of which are hereby incorporated by reference in a manner that is consistent herewith.

The absorbent body 4 of the present invention includes, but is not limited to the absorbent body liner 5, the absorbent core 3, the tissue wrapsheet 60 and the surge management layer 7. The absorbent body liner 5 forms a housing about the absorbent core 3, the tissue wrapsheet 60 and surge management layer 7. The absorbent body liner 5, the tissue wrapsheet 60 and the absorbent core 3 of the absorbent body 4 desirably comprise non-stretch material and/or low stretch material. Non-stretch materials are less expensive than stretchable material and are less difficult to process in manufacture.

FIG. 2 illustrates a cross-section view of one embodiment of the diaper 1 assembly of the present invention. The absorbent core 3 is encompassed by the tissue wrapsheet 60 such that the oppositely facing surfaces of the absorbent core 3 are contained by the tissue wrapsheet 60. The absorbent core 3 is sandwiched between the absorbent body liner 5 and the chassis 2. The chassis liner 10 is laminated to the outer cover 17 wherein the chassis liner 10 and outer cover 17 combined comprise the chassis 2. The outer cover 17 and the chassis liner 10 are laminated to each other employing various types of suitable attachment means known to one skilled in the art such as with adhesives, sonic bonding, thermal bonding, or a combination thereof. The surge management layer 7 of the present invention is preferably disposed between the absorbent body liner 5 and the absorbent core 3.

The configuration of the present invention provides a diaper 1 that is less costly and less complicated to produce. The absorbent body liner 5 houses the absorbent core 3, the tissue wrapsheet 60 and surge management layer 7 forming the absorbent body 4 which may be assembled separately from the chassis 2 and then affixed to the chassis 2. The separate assembly of the absorbent body 4 and the chassis 2 allow the diaper 1 of the present invention to be manufactured more economically by simplifying the manufacturing process as the non-stretch components of the absorbent body 4 are not integrated into the stretchable components of the chassis 2. The non-stretchable and/or low stretch absorbent body liner 5, a tissue wrapsheet 60 and the absorbent core 3 of the absorbent body 4 allow the diaper 1 construction to be cost effective as these components, if stretchable, may be more costly to manufacture and assemble. Further, by utilizing a stretchable chassis 2, the diaper 1 does not sacrifice the comfort, fit and containment qualities desirable of stretchable diaper constructions.

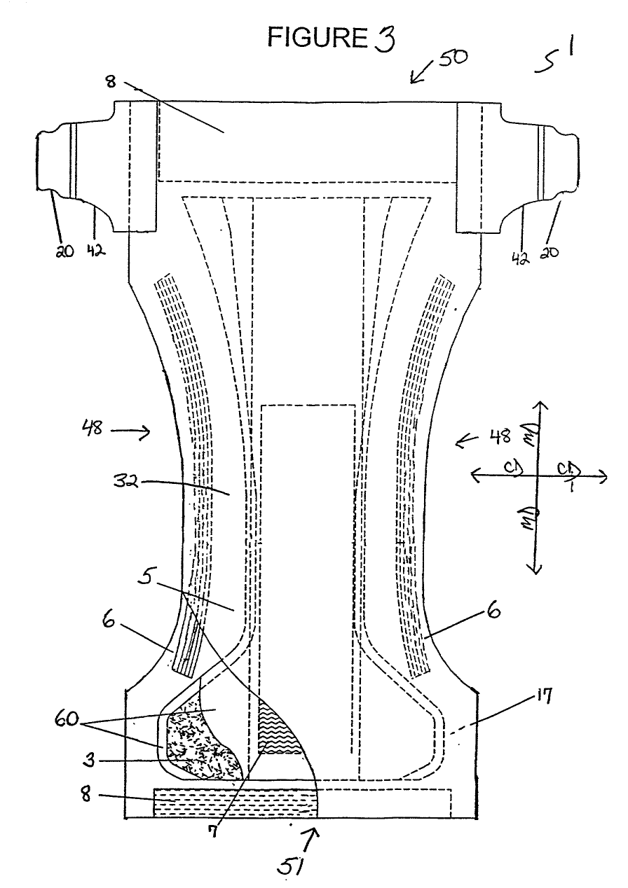

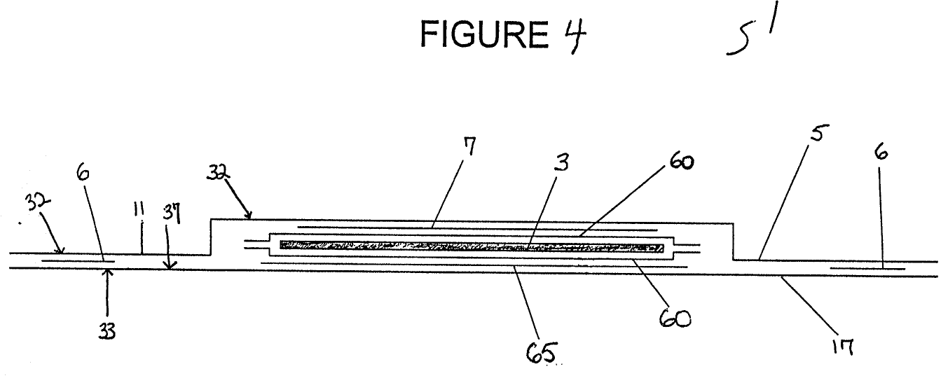

The above advantages of the diaper 1 configuration of the present invention may be illustrated by comparing the diaper 1 configuration of a conventional diaper depicted in FIGS. 3 and 4. FIG. 3 is a top cut-away view of one embodiment of a typical diaper assembly and FIG. 4 a cross-section view of one embodiment of a typical diaper 1 assembly. The diaper 1 generally defines a front waist region 51 and a back waist region 50 which together define a three-dimensional diaper 1 configuration having a waist opening and a pair of leg openings (not shown). The diaper 1 components such as the leg elastic members 6 may be interposed between the outer cover 17 and the bodyside liner 5. The fasteners 20 are assembled and attached to extend from the side panels 42 that are attached to the laterally opposed side edges in the back waist region 50 of the diaper 1. The fasteners 20 may be affixed to the inner surface 11 of the chassis 2, the inner surface 37 of the outer cover 17, or the outer surface 33 of the chassis 2. Other components such as the waist elastic members 8 may be affixed to the inner surface 32 of the bodyside liner 5. The ventilation layer 65 is located between the bodyside liner 5 and the outer cover 17 to insulate the outer cover 17 from the absorbent core 3 and to reduce the dampness of the outer surface 33 of the outer cover 17. The various layers and components of the diaper 1 are integrally assembled employing various types of suitable attachment means described above that are well know to those skilled in the art such as with adhesives, sonic bonding, thermal bonding or a combination thereof.

The outer cover 17 and the bodyside liner 5 are assembled to each other with the absorbent core 3 sandwiched therebetween. The absorbent core 3, which may be stretchable, is housed by the tissue wrapsheet 60 such that the oppositely facing surfaces of the absorbent core 3 are contained by the tissue wrapsheet 60. The surge management layer 7, which is typically stretchable, is disposed between the bodyside liner 5 and the absorbent core 3.

The configuration of the diaper 1 in FIGS. 3 and 4 is more costly and complicated to produce. The diaper 1 configuration in FIGS. 3 and 4 may utilize a multitude of stretchable elastomeric components in its construction, such as the bodyside liner 5, the absorbent core 3, the surge management layer 7 and the outer cover 17 which may be more difficult to process during manufacturing. Typically the manufacturing equipment of a conventional stretchable diaper 1 assembly requires that one or more of its stretchable layers are pre-stretched before securing another stretchable layer to the first stretchable layer(s).

For example, the diaper 1 in FIGS. 3 and 4 include an absorbent core 3 that is sandwiched between a stretchable bodyside liner 5 and a stretchable outer cover 17. The conventional diaper 1 may require that the outer cover 17 be pre-stretched before the absorbent core 3 is affixed to the outer cover 17. Similarly the bodyside liner 5 may also have to be prestretched before it is affixed to the outer cover 17 sandwiching the absorbent core 3 therebetween. Further, the more stretchable components utilized in the diaper 1 construction, the more costly it is to manufacture the diaper 1.

The diaper 1 of the present invention may be very simple to manufacture on a converting line utilizing an absorbent body 4 and a stretchable chassis 2 that are each manufactured separately. The stretchable material of the chassis 2 may easily move down a converting line in an unstretched configuration wherein the pre-assembled absorbent body 4 would simply be cut, placed and attached to the unstretched chassis 2. Other components such as the leg elastic members 6 and the waist elastic members 8 which already possess stretchable characteristics may simply be applied to the stretchable chassis 2 material without pre-stretching.

The absorbent body 4 of the diaper 1 and the stretchable chassis 2 of the diaper 1 are integrally assembled together employing various types of suitable attachment means as are well known to those skilled in the art. For example, the absorbent body 4 may be connected to the chassis 2 with an adhesive. The adhesive may be applied as a uniform continuous layer of adhesive, a patterned layer of adhesive, a sprayed pattern of adhesive, or any of separate lines, swirls or dots of adhesive. Alternatively, the absorbent body 4 may be attached to the chassis 2 using ultra sonic bonding, thermal bonding, or the like. In another alternative, the absorbent body 4 may be attached to the chassis 2 by using conventional fasteners such as buttons, hook and loop type fasteners, adhesive tape fasteners, and the like.

The non-stretch and/or low stretch nature of the absorbent body 4 creates drag to the biaxial stretchable surface area of the chassis 2 at the points of fixation of the inner surface 9 of the absorbent body 4 to the inner surface 11 of the chassis 2. For example, if the entire inner surface 9 of the absorbent body 4 is affixed or laminated to the inner surface 11 of the chassis 2, then the stretchable capacity of the surface area of the chassis 2 is reduced by the entire surface area of the inner surface 9 of the absorbent body 4. Accordingly, it is desirable that the absorbent body 4 of the present invention, including, but not limited to the tissue wrapsheet 60, the absorbent core 3 and the absorbent body liner 5 has a lower surface area than the surface area of the chassis 2 such that the perimeter and a portion of the surface area of the chassis 2 extends beyond the perimeter and the surface area of the absorbent body 4 forming a generally smaller rectangular shape within a larger generally larger rectangular shape configuration of the diaper 1.

Further, the absorbent body 4 may be partially or entirely attached to the stretchable chassis 2 material which is preferably capable of stretch/recovery in both the cross-machine direction and the machine direction. In view of the fact that an attachment of the entire surface area of the non-stretch and/or low stretch absorbent body 4 to the stretchable chassis 2 would greatly inhibit the biaxial stretch of the chassis 2, the surface area of the absorbent body 4 may be portionately attached to the stretchable multilayer chassis 2 in a cross-machine direction attachment pattern, in a machine direction attachment pattern, or both in a cross-machine direction attachment pattern and in a machine direction attachment pattern.

For example, the absorbent body 4 may be attached to the stretchable chassis 2 in a machine direction only line of attachment which would inhibit stretch in the machine direction alone by the portion of the surface area of the absorbent body 4 that is affixed to the stretchable chassis 2, but would allow the chassis 2 to stretch in the cross-machine direction. Conversely, a cross-machine direction only line of attachment of the absorbent body 4 to the stretchable chassis 2 would inhibit stretch in the cross-machine direction alone by the portion of the surface area of the absorbent body 4 that is affixed to the stretchable chassis 2, but would allow the chassis 2 to stretch in the machine direction. Further, the attachment of the absorbent body 4 may also cover only a fraction of the surface area of the stretchable chassis 2. For example, a 4 inch by 6 inch absorbent body 4 may be attached on only a 2 inch by 4 inch rectangular area of the stretchable chassis 2.

Specifically, the surface area attachment of the non-stretch and/or low stretch absorbent body 4 to the surface area of the stretchable multilayer chassis 2 is preferably less than about 95%, more preferably less than about 50%, and even more preferably less than about 25%.

Methods of manufacturing the disposable absorbent article of the present invention, methods of manufacturing individual components useful to make the disposable absorbent article of the present invention, as well as methods of using the disposable absorbent article of the present invention are disclosed, e.g., in U.S. Pat. Nos. 6,321,557; 6,193,701; 5,883,028; 5,595,618; 5,540,796; 5,490,846; 5,496,298; 5,486,166; 5,226,992; 5,116,662; 5,114,781; 4,965,122; 4,777,073; 4,704,116; 4,663,220; U.S. patent application Ser. Nos. 09/460,490; 09/455,513 and European Patent Application No. EP 0 217 032 and references cited therein.

Material Elongation and Deformation Tensile Test

A suitable technique for determining the amount of elongation, retractive force and or permanent deformation of a selected component or material can employ ASTM Standard Test Method D882 (Tensile Method for Tensile Properties of Thin Plastic Sheeting) dated December 1995, with the following particulars.

Equipment

1. Tensile tester capable of obtaining a peak load and equipped with an appropriate load cell. A suitable tensile testing system is a Sintech Tensile Tester, commercially available from MTS Sintech, Research Triangle Park, N.C., under the trade designation Model 1/G equipped with Sintech Testworks™ Version 3.10 Software.

2. Pneumatic-action grips having a 0.5 by 4 inch grip face.

3. Test facility having a temperature of 23+1° C., and a relative humidity of 50+2 percent.

The test sample width is perpendicular to the direction of the tensile force applied during the testing. With regard to the shown configurations, for example, the test sample “width” generally corresponds to the length-wise dimension of outer cover 17 along the longitudinal direction of the article. The initial separation of the jaws of the tensile tester is 3 inches (76.2 mm) at a tensile force of about 1 gram force per inch of width of the test sample, and the moving jaw is moved at a constant rate of 127 mm/min. The moving jaw is stopped at an extension where the tensile force equals 100 grams force per inch of width of the test sample, held at that extension for a period of 2 minutes, and then returned back to its initial tensile force of about 1 gram force per inch of width of the test sample at a rate of 127 mm/min. The percentage of stretch elongation, extension or permanent deformation can be determined in accordance with the following formula:

100*(L−LO)/(LO);

where:

L=either a) extended length for elongation or extension or b) post extended length for set or deformation, and

LO=initial length.

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled. All patents, patent documents, and references cited and disclosed herein are incorporated by reference herein.

What is claimed is:

1. A disposable absorbent article comprising:
(i) a stretchable multilayer chassis that defines an inner surface and an outer surface;
(ii) an absorbent body having an inner surface and an outer surface
wherein:
(a) the stretchable multilayer chassis is stretchable in at least the cross-machine direction;
(b) the absorbent body is affixed to the inner surface of the multilayer stretchable chassis
such that in use, the inner surface of the absorbent body lies against the wearer and the stretchable multilayer chassis stretches about the wearer independently in at least the cross-machine direction.

2. The disposable absorbent article of claim 1, which is a diaper.

3. The disposable absorbent article of claim 1, wherein the absorbent body is attached to the stretchable multilayer chassis in a cross-machine direction attachment pattern, in a machine direction attachment pattern, or both in a cross-machine direction attachment pattern and in a machine direction attachment pattern.

4. The disposable absorbent article of claim 3, wherein less than about 95% of the surface area of the absorbent body is attached to the surface area of the stretchable multilayer chassis.

5. The disposable absorbent article of claim 3, wherein less than about 50% of the surface area of the absorbent body is attached to the surface area of the stretchable multilayer chassis.

6. The disposable absorbent article of claim 3, wherein less than about 25% of the surface area of the absorbent body is attached to the surface area of the stretchable multilayer chassis.

7. The disposable absorbent article of claim 1, wherein the stretchable multilayer chassis comprises at least two layers.

8. The disposable absorbent article of claim 7, wherein the stretchable multilayer chassis comprises a chassis liner and an outer cover.

9. The disposable absorbent article of claim 8, wherein the chassis liner and the outer cover are both independently extensible.

10. The disposable absorbent article of claim 8, wherein the chassis liner and the outer cover are both independently elastic.

11. The disposable absorbent article of claim 8, wherein the chassis liner is independently extensible and the outer cover is independently elastic.

12. The disposable absorbent article of claim 8, wherein the chassis liner is independently elastic and the outer cover is independently extensible.

13. The disposable absorbent article of claim 8, wherein the chassis liner comprises a neck-stretched, spunbond web.

14. The disposable absorbent article of claim 8, wherein the outer cover comprises elastic material that is stretchable independently in both the cross-machine direction and the machine direction by at least 25% to at least 125%.

15. The disposable absorbent article of claim 8, wherein the outer cover comprises elastic material that is stretchable independently in both the cross-machine direction and the machine direction by at least 100% to at least 200%.

16. The disposable absorbent article of claim 8, wherein the outer cover comprises elastic material that is stretchable independently in both the cross-machine direction and the machine direction by at least 150% to at least 250%.

17. The disposable absorbent article of claim 8, wherein the outer cover comprises polypropylene spunbond material that is laminated with styrene-isoprene-styrene-based adhesive.

18. The disposable absorbent article of claim 8, wherein the outer cover comprises microporous polymer films, nonwoven fabrics, a spunbonded laminate, a meltblown laminate, a spunbond-meltblown-spunbond laminate, a stretch-bonded laminate, or a combination thereof.

19. The disposable absorbent article of claim 8, wherein the outer cover comprises a thermoplastic nonwoven web made from stretchable polymers.

20. The disposable absorbent article of claim 19, wherein the stretchable polymers comprise polyolefins, polyethylene, heterophasic polymers, olefin polymers, multi-block elastomeric copolymers, or a combination thereof.

21. The disposable absorbent article of claim 1, wherein the absorbent body comprises at least an absorbent core.

22. The disposable absorbent article of claim 1, wherein the non-stretch absorbent body comprises an absorbent core, a tissue wrapsheet, an absorbent body liner, or a combination thereof.

23. The disposable absorbent article of claim 22, wherein the absorbent core comprises a matrix of hydrophilic fibers and superabsorbent material.

24. A disposable absorbent article comprising:
i) an absorbent body liner that defines an inner surface and an outer surface;
ii) an absorbent core that defines an inner surface and an outer surface;
iii) a tissue wrapsheet;
iv) a surge management layer that defines an inner surface and an outer surface;
v) a chassis liner that defines an inner surface and an outer surface wherein the chassis liner is stretchable independently in both the cross-machine direction and the machine direction; and
vi) an outer cover that defines an inner surface and an outer surface wherein the outer cover is stretchable independently in both the cross-machine direction and the machine direction
wherein:
(a) the absorbent body liner houses the absorbent core, the tissue wrapsheet and the surge management layer forms an absorbent body; and
(b) the outer surface of the chassis liner is laminated to the inner surface of the outer cover forming a stretchable multilayer chassis defining an inner surface and an outer surface
such that the inner surface of the absorbent core and the outer surface of the absorbent core are wrapped by a tissue wrap sheet; the inner surface of the surge management layer is adjacent to the outer surface of the absorbent body liner and the outer surface of the surge management layer is adjacent to the tissue wrapsheet which is adjacent to the inner surface of the absorbent core; and wherein the outer surface of the absorbent body liner is affixed to the inner surface of the stretchable multilayer chassis.

25. The disposable absorbent article of claim 24 which is a diaper.

26. The disposable absorbent article of claim 24, wherein the absorbent body is attached to the stretchable multilayer chassis in a cross-machine direction attachment pattern, in a machine direction attachment pattern, or both in a cross-machine direction attachment pattern and in a machine direction attachment pattern.

27. The disposable absorbent article of claim 26, wherein less than about 95% of the surface area of the absorbent body is attached to the surface area of the stretchable multilayer chassis.

28. The disposable absorbent article of claim 26, wherein less than about 50% of the surface area of the absorbent body is attached to the surface area of the stretchable multilayer chassis.

29. The disposable absorbent article of claim 26, wherein less than about 25% of the surface area of the absorbent body is attached to the surface area of the stretchable multilayer chassis.

30. The disposable absorbent article of claim 24, wherein the chassis liner and the outer cover are both independently extensible.

31. The disposable absorbent article of claim 24, wherein the chassis liner and the outer cover are both independently elastic.

32. The disposable absorbent article of claim 24, wherein the chassis liner is independently extensible and the outer cover is independently elastic.

33. The disposable absorbent article of claim 24, wherein the chassis liner is independently elastic and the outer cover is independently extensible.

34. The disposable absorbent article of claim 24, wherein the chassis liner comprises a neck-stretched, spunbond web.

35. The disposable absorbent article of claim 24, wherein the outer cover comprises elastic material that is stretchable independently in both the cross-machine direction and the machine direction by at least 25% to at least 150% of an initial unstretched configuration.

36. The disposable absorbent article of claim 24, wherein the outer cover comprises elastic material that is stretchable independently in both the cross-machine direction and the machine direction by at least 100% to at least 200% of an initial unstretched configuration.

37. The disposable absorbent article of claim 24, wherein the outer cover comprises elastic material that is stretchable independently in both the cross-machine direction and the machine direction by at least 150% to at least 250% of an initial unstretched configuration.

38. The disposable absorbent article of claim 24, wherein the outer cover comprises a polypropylene spunbond that is laminated with styrene-isoprene-styrene-based adhesive.

39. The disposable absorbent article of claim 24, wherein the outer cover comprises microporous polymer films, nonwoven fabrics, a spunbonded laminate, a meltblown laminate, a spunbond-meltblown-spunbond laminate, a stretch-bonded laminate, or a combination thereof.

40. The disposable absorbent article of claim 24, wherein the outer cover comprises a thermoplastic nonwoven web made from stretchable polymers.

41. The disposable absorbent article of claim 40, wherein the stretchable polymers comprise polyolefins, polyethylene, heterophasic polymers, olefin polymers, multi-block elastomeric copolymers, or a combination thereof.

42. The disposable absorbent article of claim 24, wherein the absorbent body comprises non-stretch material, low stretch material, or a combination thereof.

43. The disposable absorbent article of claim 24, wherein the absorbent core comprises a matrix of hydrophilic fibers and superabsorbent fibers.
